(19) United States
(12) Patent Application Publication
(10) Pub. No.: US 2025/0189399 A1
(43) Pub. Date: Jun. 12, 2025

(54) GAS LEAK DETECTION DEVICE

(71) Applicants: Hyundai Motor Company, Seoul (KR); Kia Corporation, Seoul (KR)

(72) Inventor: Byung Ryeol Lee, Yongin-si (KR)

(21) Appl. No.: 18/608,105

(22) Filed: Mar. 18, 2024

(30) Foreign Application Priority Data
Dec. 7, 2023 (KR) 10-2023-0177102

(51) Int. Cl. G01M 3/04

(52) U.S. Cl. CPC G01M 3/04

(57) ABSTRACT

An embodiment gas leak detection device includes a tip head part having a spherical shape, a tip connection part configured to communicate with the tip head part and connectable to a hydrogen detection line, a reference detection hole disposed at an end of the tip head part based on a longitudinal direction of the tip connection part, and a peripheral detection hole disposed in the tip head part, spaced apart from the reference detection hole, and directed in a direction intersecting the reference detection hole.

### CROSS-REFERENCE TO RELATED APPLICATIONS

This application claims the benefit of Korean Patent Application No. 10-2023-0177102, filed on Dec. 7, 2023, which application is hereby incorporated herein by reference.

### TECHNICAL FIELD

The present disclosure relates to a gas leak detection device.

### BACKGROUND

A fuel cell electric vehicle (FCEV) produces electrical energy from an electrochemical reaction between oxygen and hydrogen in a fuel cell stack and travels by operating a motor.

The fuel cell electric vehicle may continuously generate electricity, regardless of a capacity of a battery, by being supplied with fuel (hydrogen) and air from the outside, and thus has high efficiency and emits almost no contaminant. By virtue of these advantages, continuous research and development is being conducted on the fuel cell electric vehicle.

In general, the fuel cell electric vehicle may include a fuel cell stack configured to generate electricity by means of an oxidation-reduction reaction between hydrogen and oxygen, a fuel supply device configured to supply fuel (hydrogen) to the fuel cell stack, and an air supply device configured to supply the fuel cell stack with reaction air (oxygen) which is an oxidant required for an electrochemical reaction.

Meanwhile, a sealing performance of connecting parts (e.g., a regulator, a hydrogen shut-off valve, a hydrogen supply valve, and fitting parts for pipes) in the hydrogen supply line for supplying the hydrogen in the fuel cell electric vehicle is one of the most important performances related to the safety of a hydrogen supply system, and particularly, the safety of the entire fuel cell system.

In particular, secondary damage such as a fire may occur when the hydrogen leaks from the connecting parts in the hydrogen supply line.

The risk of the occurrence of a safety accident increases when hydrogen leaks from the connecting parts in the hydrogen supply line. Therefore, it is necessary to accurately detect whether hydrogen leaks.

Therefore, in the related art, there has been proposed a method of detecting a leak of hydrogen by allowing a detection tip, which has an approximately pipe shape and is provided at an end of a hydrogen detector, to approach an inspection site (a site where a leak of hydrogen is expected).

The detection tip needs to approach (be disposed at) the inspection site in an accurate posture (angle and distance) in order to improve the accuracy of the hydrogen leak inspection performed by the hydrogen detector. However, because hydrogen has a low gas density and colorless and odorless properties, it is difficult for an operator to accurately identify a hydrogen leak point, which makes it difficult to allow the detection tip to approach the inspection site in an accurate posture (angle and distance). Further, there is a problem in that a deviation in the accuracy of the hydrogen leak inspection greatly varies depending on the operator's experience and competence.

In addition, in the related art, because a leak rate of hydrogen, which is detected by the hydrogen detector, varies depending on the posture of the detection tip with respect to the inspection site, there is a problem in that it is difficult to quantify the inspection result.

Therefore, recently, various studies have been conducted to simplify a hydrogen leak detection process and improve the accuracy of a hydrogen leak inspection, but the study results are still insufficient. Accordingly, there is a need to develop a technology to simplify the hydrogen leak detection process and improve the accuracy of the hydrogen leak inspection.

### SUMMARY

The present disclosure relates to a gas leak detection device. Particular embodiments relate to a gas leak detection device capable of improving the accuracy of a hydrogen leak inspection and improving safety and reliability.

Embodiments of the present disclosure can provide a gas leak detection device capable of improving the accuracy of a hydrogen leak inspection and improving safety and reliability.

In particular, embodiments of the present disclosure can minimize a deviation in the accuracy of the hydrogen leak inspection, which varies depending on an operator's experience and competence, and can easily and accurately detect whether hydrogen leaks.

Among other things, embodiments of the present disclosure can accurately perform the hydrogen leak inspection without being greatly affected by a posture (angle and distance) of a detection tip with respect to an inspection site.

Embodiments of the present disclosure can simplify a hydrogen leak inspection process and shorten the time required for the inspection process.

Embodiments of the present disclosure can minimize an error in the hydrogen leak inspection and quantify a result of the hydrogen leak inspection.

The features achievable by the embodiments are not limited to the above-mentioned features, but the embodiments also include other features or effects that may be understood from the solutions or embodiments described below.

An embodiment of the present disclosure provides a gas leak detection device including a tip head part having a spherical shape, a tip connection part configured to communicate with the tip head part and connected to a hydrogen detection line, a reference detection hole provided at an end of the tip head part based on a longitudinal direction of the tip connection part, and a peripheral detection hole provided in the tip head part, spaced apart from the reference detection hole, and directed in a direction intersecting the reference detection hole.

This may improve the accuracy of a hydrogen leak inspection and improve the safety and reliability.

That is, the detection tip needs to approach (be disposed at) the inspection site in an accurate posture (angle and distance) in order to improve the accuracy of the hydrogen leak inspection performed by the hydrogen detector. However, because hydrogen has a low gas density and colorless and odorless properties, it is difficult for an operator to accurately identify a hydrogen leak point, which makes it difficult to allow the detection tip to approach the inspection site in an accurate posture (angle and distance). Further, there is a problem in that a deviation in the accuracy of the hydrogen leak inspection greatly varies depending on the operator's experience and competence.

In addition, in the related art, because a leak rate of hydrogen, which is detected by the hydrogen detector, varies depending on the posture of the detection tip with respect to the inspection site, there is a problem in that it is difficult to quantify the inspection result.

In contrast, in an embodiment of the present disclosure, because the peripheral detection hole is provided in the tip head part together with the reference detection hole, the effective detection region (hydrogen leak detection region) of the tip head part may be further expanded. Therefore, it is possible to obtain an advantageous effect of improving the accuracy of the hydrogen leak inspection and improving the safety and reliability.

In particular, in an embodiment of the present disclosure, because the peripheral detection holes are provided around the reference detection hole, it is possible to obtain an advantageous effect of minimizing a deviation in the accuracy of the hydrogen leak inspection, which varies depending on the operator's experience and competence, and easily and accurately detecting whether hydrogen leaks.

Among other things, in an embodiment of the present disclosure, the leak of hydrogen may be detected through the reference detection hole and the peripheral detection holes even in a state in which the reference detection hole is not accurately consistent with the leak point. Further, it is possible to accurately perform the hydrogen leak inspection without being greatly affected by a posture (angle and distance) of the detection tip with respect to the inspection site.

According to an exemplary embodiment of the present disclosure, a center of the peripheral detection hole may be defined to be positioned on a first reference line defined to be inclined at a preset first reference angle with respect to a centerline that passes through a center of the tip head part and a center of the reference detection hole.

The first reference angle may be variously changed in accordance with required conditions and design specifications. According to an exemplary embodiment of the present disclosure, the first reference angle may be defined to be 45 degrees or less. In particular, the first reference angle may be defined as 30 degrees.

The number of peripheral detection holes and the arrangement pattern of the peripheral detection holes may be variously changed in accordance with required conditions and design specifications.

According to an exemplary embodiment of the present disclosure, the peripheral detection hole is provided as a plurality of peripheral detection holes spaced apart from one another in a circumferential direction based on the centerline (based on the reference detection hole).

According to an exemplary embodiment of the present disclosure, the tip head part may be defined to have a diameter of 20 mm and a thickness of 2 mm, a diameter of each of the reference detection hole and the peripheral detection hole may be defined as 4 mm, and the peripheral detection holes may be provided as seven peripheral detection holes spaced apart from one another at equal intervals in a circumferential direction based on the centerline.

According to an exemplary embodiment of the present disclosure, the reference detection hole and the peripheral detection hole may be defined to be positioned in a tip detection zone defined on a tip portion of the tip head part based on the longitudinal direction of the tip connection part.

According to an exemplary embodiment of the present disclosure, the tip detection zone may be defined to be 15% or less of an overall area of the tip head part.

According to an exemplary embodiment of the present disclosure, a total sum of opening areas of the reference detection hole and the peripheral detection hole may be defined to be 55% or more of an area of the tip detection zone.

According to an exemplary embodiment of the present disclosure, the gas leak detection device may include side detection holes provided in the tip head part and positioned between the peripheral detection hole and the tip connection part.

This is to detect and determine whether hydrogen leaks from the detection target (hydrogen leak site) (to measure a gas leak value) even in a state in which the tip detection zone of the tip head part, in which the reference detection hole and the peripheral detection holes are provided, is not disposed (aligned) in a posture directed toward the detection target.

That is, in case that the reference detection hole and the peripheral detection holes are provided only in the tip detection zone of the tip head part, there is a problem in that it is difficult to detect a leak of hydrogen at a lateral or rear portion of the tip head part (a lateral or rear portion of the tip head part based on the tip of the tip head part based on the longitudinal direction of the tip connection part).

In contrast, in an embodiment of the present disclosure, because the side detection holes are provided between the peripheral detection holes and the tip connection part, it is possible to detect whether hydrogen leaks from the detection target on the basis of the hydrogen introduced into the side detection holes at the lateral or rear portion of the tip head part even though the tip detection zone of the tip head part is not accurately aligned with the detection target (a direction in which hydrogen leaks from the detection target is not consistent with the reference detection hole).

The side detection hole may have various structures in accordance with required conditions and design specifications.

According to an exemplary embodiment of the present disclosure, the side detection holes may include a first side hole provided between the peripheral detection hole and the tip connection part and a second side hole provided between the first side hole and the tip connection part and spaced apart from the first side hole.

According to an exemplary embodiment of the present disclosure, a center of the first side hole may be defined to be positioned on a second reference line defined to be inclined at a preset second reference angle with respect to a centerline that passes through a center of the tip head part and a center of the reference detection hole, and a center of the second side hole may be defined to be positioned on a third reference line defined to be inclined at a preset third reference angle with respect to the centerline that passes through the center of the tip head part and the center of the reference detection hole.

The second reference angle and the third reference angle may be variously changed in accordance with required conditions and design specifications.

According to an exemplary embodiment of the present disclosure, the second reference angle may be defined as 90 degrees, and the third reference angle may be defined as 120 degrees.

The first and second side holes may be variously changed in numbers and arrangement patterns in accordance with required conditions and design specifications.

According to an exemplary embodiment of the present disclosure, the tip head part may be defined to have a diameter of 20 mm and a thickness of 2 mm, a diameter of each of the first side hole and the second side hole may be defined as 4 mm, the first side holes may be provided as four first side holes spaced apart from one another at equal intervals in the circumferential direction based on the centerline, and the second side holes may be provided as three second side holes spaced apart from one another at equal intervals in the circumferential direction based on the centerline.

A ratio of a total sum of opening areas of the reference detection hole, the peripheral detection holes, and the side detection holes to the overall area of the tip head part (the overall outer surface area of the tip head part) may be variously changed in accordance with required conditions and design specifications.

According to an exemplary embodiment of the present disclosure, a total sum of opening areas of the reference detection hole, the peripheral detection hole, and the side detection hole may be defined to be less than 15% of an overall area of the tip head part.

This is based on the fact that when the total sum of opening areas of the reference detection hole, the peripheral detection holes, and the side detection holes is equal to or more than 15% of the overall area of the tip head part, the measurement dispersion related to the leak rate of hydrogen (gas leak value) is increased by an external influence (e.g., outside air), which degrades the detection accuracy. According to an embodiment of the present disclosure, because the total sum of opening areas of the reference detection hole, the peripheral detection holes, and the side detection holes is defined to be less than 15% of the overall area of the tip head part, it is possible to obtain an advantageous effect of reducing the measurement dispersion related to the leak rate of hydrogen (gas leak value) caused by the external influence and improving the detection accuracy.

In particular, the total sum of opening areas of the reference detection hole, the peripheral detection holes, and the side detection holes may be defined as 14% of the overall area of the tip head part.

According to an exemplary embodiment of the present disclosure, a negative pressure may be applied to the hydrogen detection line, and a suction pressure may be applied to the reference detection hole, the peripheral detection hole, and the side detection hole on the basis of the negative pressure.

A suction rate (a suction rate implemented by the suction pressure) through the reference detection hole, the peripheral detection hole, and the side detection hole may be variously changed in accordance with required conditions and design specifications.

According to an exemplary embodiment of the present disclosure, a suction rate through the reference detection hole, the peripheral detection hole, and the side detection hole may be defined as 3,000 sccm.

### BRIEF DESCRIPTION OF THE DRAWINGS

FIG. 1 is a view for explaining a gas leak detection device according to an embodiment of the present disclosure.

FIG. 2 is a view for explaining a tip head part and a tip connection part of the gas leak detection device according to an embodiment of the present disclosure.

FIGS. 3 to 5 are views for explaining a reference detection hole, peripheral detection holes, and side detection holes of the gas leak detection device according to an embodiment of the present disclosure.

FIG. 6 is a view for explaining Comparative Example 1 of the gas leak detection device according to an embodiment of the present disclosure.

FIG. 7 is a view for explaining Comparative Example 2 of the gas leak detection device according to an embodiment of the present disclosure.

FIG. 8 is a view for explaining Comparative Example 3 of the gas leak detection device according to an embodiment of the present disclosure.

FIG. 9 is a view for explaining Comparative Example 4 of the gas leak detection device according to an embodiment of the present disclosure.

FIG. 10 is a view for explaining gas leak values of the comparative examples in accordance with the number of detection holes and the sizes of the detection holes.

FIG. 11 is a view for explaining gas leak rates of the comparative examples in accordance with the number of detection holes and the sizes of the detection holes.

FIG. 12 is a view for explaining gas leak rates of embodiments of the present disclosure in accordance with detection angles with respect to leak points.

FIG. 13 is a view for explaining gas leak rates of embodiments of the present disclosure in accordance with the number of detection holes under a low-suction rate condition.

FIG. 14 is a view for explaining gas leak rates of the comparative examples in accordance with the sizes of the detection holes under a low-suction rate condition and a high-suction rate condition.

FIG. 15 is a view for explaining gas leak rates of embodiments of the present disclosure in accordance with suction rates.

### DETAILED DESCRIPTION OF ILLUSTRATIVE EMBODIMENTS

Hereinafter, exemplary embodiments of the present disclosure will be described in detail with reference to the accompanying drawings.

However, the technical spirit of the present disclosure is not limited to the embodiments described herein but may be implemented in various different forms. One or more of the constituent elements in the embodiments may be selectively combined and substituted for use within the scope of the technical spirit of the present disclosure.

In addition, unless otherwise specifically and explicitly defined and stated, the terms (including technical and scientific terms) used in the embodiments of the present disclosure may be construed as having the meaning which may be commonly understood by the person with ordinary skill in the art to which the present disclosure pertains. The meanings of the commonly used terms such as the terms defined in dictionaries may be interpreted in consideration of the contextual meanings of the related technology.

In addition, the terms used in the embodiments of the present disclosure are for explaining the embodiments, not for limiting the present disclosure.

In the present specification, unless particularly stated otherwise, a singular form may also include a plural form. The expression “at least one (or one or more) of A, B, and C” may include one or more of all combinations that can be made by combining A, B, and C.

In addition, the terms such as first, second, A, B, (a), and (b) may be used to describe constituent elements of the embodiments of the present disclosure.

These terms are used only for the purpose of discriminating one constituent element from another constituent element, and the nature, the sequences, or the orders of the constituent elements are not limited by the terms.

Further, when one constituent element is described as being ‘connected’, ‘coupled’, or ‘attached’ to another constituent element, one constituent element may be connected, coupled, or attached directly to another constituent element or connected, coupled, or attached to another constituent element through still another constituent element interposed therebetween.

In addition, the expression “one constituent element is provided or disposed above (on) or below (under) another constituent element” includes not only a case in which the two constituent elements are in direct contact with each other, but also a case in which one or more other constituent elements are provided or disposed between the two constituent elements. The expression “above (on) or below (under)” may mean a downward direction as well as an upward direction based on one constituent element.

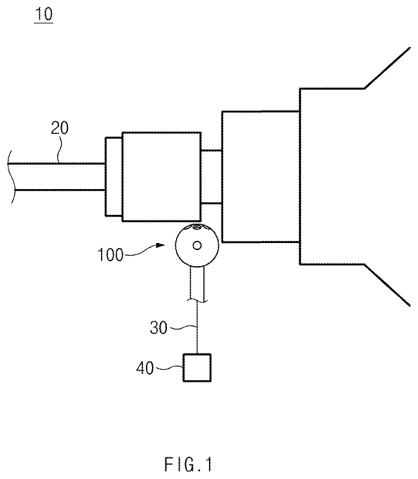

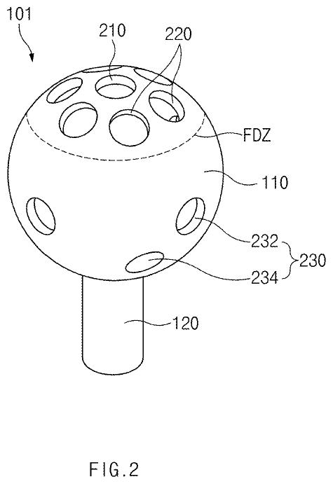

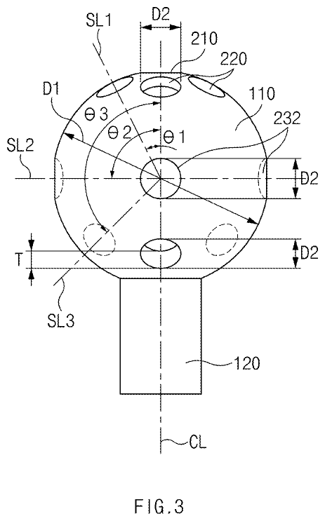

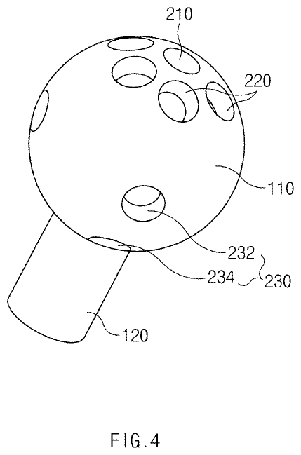

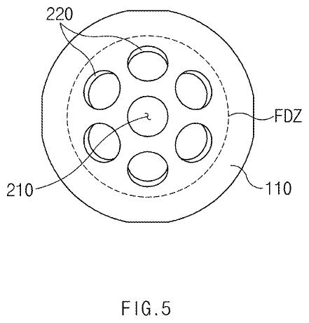

With reference to FIGS. 1 to 15, a gas leak detection device 10 according to an embodiment of the present disclosure includes a tip head part 110 having a spherical shape, a tip connection part 120 configured to communicate with the tip head part 110 and connected to a hydrogen detection line 30, a reference detection hole 210 provided at an end of the tip head part 110 based on a longitudinal direction of the tip connection part 120, and peripheral detection holes 220 provided in the tip head part 110, spaced apart from the reference detection hole 210, and directed in a direction intersecting the reference detection hole 210.

For reference, the gas leak detection device 10 according to an embodiment of the present disclosure may detect a leak of a gas. Embodiments of the present disclosure are not restricted or limited by the type and properties of the gas detected by the gas leak detection device 10.

Hereinafter, an example will be described in which the gas leak detection device 10 according to an embodiment of the present disclosure is used to detect a leak of hydrogen from a connecting part of a hydrogen supply line 20 through which hydrogen is supplied.

The tip head part 110 and the tip connection part 120 collectively constitute a detection tip 100. The hydrogen introduced into the tip head part 110 may be introduced into a hydrogen detection part 40 along the hydrogen detection line 30 connected to an end of the tip connection part 120. The hydrogen detection part 40 may determine whether the hydrogen leaks on the basis of the amount of introduced hydrogen.

Various hydrogen detection sensors capable of detecting hydrogen may be used as the hydrogen detection part 40. Embodiments of the present disclosure are not restricted or limited by the type and structure of the hydrogen detection sensor.

For example, a semiconductor element sensor, a ceramic sensor, an electrochemical sensor, an optical sensor, or the like may be used as the hydrogen detection sensor.

The tip head part 110 is configured to come into direct contact with a detection target (hydrogen leak site).

The tip head part 110 has a hollow spherical shape (ball shape). Embodiments of the present disclosure are not restricted or limited by the size (diameter and thickness) D1 of the tip head part 110.

According to an exemplary embodiment of the present disclosure, a diameter D1 of the tip head part 110 may be defined as 20 mm, and a thickness T of the tip head part 110 may be defined as 2 mm. According to another embodiment of the present disclosure, the diameter of the tip head part may be smaller or larger than 20 mm, and the thickness of the tip head part may be smaller or larger than 2 mm.

The tip connection part 120 communicates with the tip head part 110, and the hydrogen detection line 30 is connected to one end of the tip connection part 120.

The tip connection part 120 may have various structures capable of communicating with the tip head part 110. Embodiments of the present disclosure are not restricted or limited by the structure of the tip connection part 120.

For example, the tip connection part 120 may have an approximately hollow cylindrical shape. The tip connection part 120 is accommodated in a fastening hole (not illustrated) provided in the tip head part 110, such that the tip connection part 120 may be connected to the tip head part 110 while communicating with the tip head part 110.

In the embodiment of the present disclosure illustrated and described above, an example is described in which the tip connection part 120 is connected directly to the tip head part 110. However, according to another embodiment of the present disclosure, the tip connection part may be connected to the tip head part by means of a separate adapter. Alternatively, the tip connection part may be screw-fastened to the tip head part. Alternatively, the tip connection part may be fastened to the tip head part by means of a separate clip or fastening member.

The reference detection hole 210 may be provided at a distal tip of the tip head part 110 based on the longitudinal direction of the tip connection part 120. The hydrogen leaking from the detection target (hydrogen leak site) may be introduced into the tip head part 110 through the reference detection hole 210.

For example, a center of the reference detection hole 210 is positioned on a centerline CL that passes through a center of the tip connection part 120 and a center of the tip head part 110.

The reference detection hole 210 may have various structures in accordance with required conditions and design specifications. Embodiments of the present disclosure are not restricted or limited by the structure and shape of the reference detection hole 210.

For example, the reference detection hole 210 may be provided in the form of a circular hole. Alternatively, the reference detection hole 210 may have a quadrangular shape, an elliptical shape, or other shapes.

A size (diameter) D2 of the reference detection hole 210 may be variously changed in accordance with required conditions and design specifications. Embodiments of the present disclosure are not restricted or limited by the size of the reference detection hole 210.

According to an exemplary embodiment of the present disclosure, a diameter D2 of the reference detection hole 210 may be defined as 4 mm. According to another embodiment of the present disclosure, the diameter of the reference detection hole may be smaller than 4 mm or larger than 4 mm.

The peripheral detection holes 220 are provided around the tip head part 110, spaced apart from the reference detection hole 210, and directed in the direction intersecting the reference detection hole 210. The hydrogen leaking from the detection target (hydrogen leak site) may be introduced into the tip head part 110 through the peripheral detection holes 220.

A leak rate of hydrogen from the detection target may be substantially determined on the basis of the hydrogen introduced through the reference detection hole 210 and the peripheral detection holes 220.

As described above, in an embodiment of the present disclosure, the leak rate of hydrogen from the detection target is detected on the basis of the hydrogen introduced through the reference detection hole 210 and the hydrogen introduced through the peripheral detection holes 220, such that an effective detection region (hydrogen leak detection region) of the tip head part 110 may be further expanded. Therefore, it is possible to obtain an advantageous effect of improving the accuracy of the hydrogen leak inspection and improving the safety and reliability.

In particular, in an embodiment of the present disclosure, the leak of hydrogen may be detected through the reference detection hole 210 and the peripheral detection holes 220 even in a state in which the reference detection hole 210 is not accurately consistent with the leak point. Therefore, it is possible to minimize a deviation in the accuracy of the hydrogen leak inspection that varies depending on the operator's experience and competence. Further, it is possible to accurately perform the hydrogen leak inspection without being greatly affected by a posture (angle and distance) of the detection tip 100 with respect to the inspection site.

Moreover, in an embodiment of the present disclosure, the hydrogen leaking from the detection target is divided and introduced into the reference detection hole 210 and the peripheral detection holes 220. Therefore, it is possible to obtain an advantageous effect of minimizing the dispersion of gas leak values and standardizing (quantifying) the measurement values.

The peripheral detection hole 220 may have various structures in accordance with required conditions and design specifications. Embodiments of the present disclosure are not restricted or limited by the structure and shape of the peripheral detection hole 220.

For example, the peripheral detection hole 220 may be provided in the form of a circular hole. Alternatively, the peripheral detection hole 220 may have a quadrangular shape, an elliptical shape, or other shapes.

A size (diameter) of the peripheral detection hole 220 may be variously changed in accordance with required conditions and design specifications. Embodiments of the present disclosure are not restricted or limited by the size of the peripheral detection hole 220.

According to an exemplary embodiment of the present disclosure, a diameter of the peripheral detection hole 220 may be defined as 4 mm. According to another embodiment of the present disclosure, the diameter of the peripheral detection hole may be smaller than 4 mm or larger than 4 mm.

According to an exemplary embodiment of the present disclosure, a center of the peripheral detection hole 220 may be positioned on a first reference line SL1 inclined at a preset first reference angle θ1 with respect to the centerline CL that passes through the center of the tip head part 110 and the center of the reference detection hole 210.

The first reference angle θ1 may be variously changed in accordance with required conditions and design specifications. Embodiments of the present disclosure are not restricted or limited by the size of the first reference angle θ1.

According to an exemplary embodiment of the present disclosure, the first reference angle θ1 may be defined as 45 degrees or less. In particular, the first reference angle θ1 may be defined as 30 degrees.

The peripheral detection hole 220 may be variously changed in number and arrangement pattern in accordance with required conditions and design specifications. Embodiments of the present disclosure are not restricted or limited by the number and arrangement pattern of the peripheral detection holes 220.

According to an exemplary embodiment of the present disclosure, the plurality of peripheral detection holes 220 may be provided to be radially disposed and spaced apart from one another in a circumferential direction based on the centerline CL (based on the reference detection hole 210).

In particular, seven peripheral detection holes 220 may be provided to be spaced apart from one another at equal intervals in the circumferential direction based on the centerline CL.

According to another embodiment of the present disclosure, the number of peripheral detection holes may be defined to be smaller than seven or equal to or larger than eight.

According to an exemplary embodiment of the present disclosure, the reference detection hole 210 and the peripheral detection holes 220 may be positioned in a tip detection zone FDZ defined on a tip portion of the tip head part 110 based on the longitudinal direction of the tip connection part 120.

The tip detection zone FDZ may be defined in various shapes in accordance with required conditions and design specifications. Embodiments of the present disclosure are not restricted or limited by the shape of the tip detection zone FDZ.

For example, the tip detection zone FDZ may be defined in an approximately dome shape.

In particular, a center of the dome of the tip detection zone FDZ may be defined to be consistent with the center of the reference detection hole 210.

According to an exemplary embodiment of the present disclosure, the tip detection zone FDZ may be defined to be 15% or less of an overall area of the tip head part 110 (an overall outer surface area of the tip head part 110). In particular, the tip detection zone FDZ may be defined to be 12.3% of the overall area of the tip head part 110 (the overall outer surface area of the tip head part 110).

According to an exemplary embodiment of the present disclosure, a total sum of an opening area of the reference detection hole 210 and opening areas of the peripheral detection holes 220 (e.g., a total sum of an opening area of one reference detection hole and opening areas of six peripheral detection holes) may be defined to be 55% or more of the area of the tip detection zone FDZ.

In particular, the total sum of the opening area of the reference detection hole 210 and the opening areas of the peripheral detection holes 220 may be defined to be 57.1% of the area of the tip detection zone FDZ.

According to an exemplary embodiment of the present disclosure, the gas leak detection device 10 may include side detection holes 230 provided in the tip head part 110 and positioned between the peripheral detection holes 220 and the tip connection part 120.

The side detection holes 230 are provided to detect and determine whether hydrogen leaks from the detection target (hydrogen leak site) (to measure a gas leak value) even in a state in which the tip detection zone FDZ of the tip head part 110, in which the reference detection hole 210 and the peripheral detection holes 220 are provided, is not disposed (aligned) in a posture directed toward the detection target.

That is, in case that the reference detection hole 210 and the peripheral detection holes 220 are provided only in the tip detection zone FDZ of the tip head part 110, there is a problem in that it is difficult to detect a leak of hydrogen at a lateral or rear portion of the tip head part 110 (a lateral or rear portion of the tip head part 110 based on the tip of the tip head part 110 based on the longitudinal direction of the tip connection part 120).

In contrast, in an embodiment of the present disclosure, because the side detection holes 230 are provided between the peripheral detection holes 220 and the tip connection part 120, it is possible to detect whether hydrogen leaks from the detection target on the basis of the hydrogen introduced into the side detection holes 230 at the lateral or rear portion of the tip head part 110 even though the tip detection zone FDZ of the tip head part 110 is not accurately aligned with the detection target (a direction in which hydrogen leaks from the detection target is not consistent with the reference detection hole 210).

The side detection holes 230 may be substantially used to determine whether hydrogen leaks from the detection target. The leak rate of hydrogen (gas leak value) from the detection target may be detected on the basis of the hydrogen introduced through the reference detection hole 210 and the peripheral detection holes 220.

The side detection hole 230 may have various structures in accordance with required conditions and design specifications. Embodiments of the present disclosure are not restricted or limited by the structure and shape of the side detection hole 230.

For example, the side detection hole 230 may be provided in the form of a circular hole. Alternatively, the side detection hole 230 may have a quadrangular shape, an elliptical shape, or other shapes.

A size (diameter) of the side detection hole 230 may be variously changed in accordance with required conditions and design specifications. Embodiments of the present disclosure are not restricted or limited by the size of the side detection hole 230.

According to an exemplary embodiment of the present disclosure, a diameter of the side detection hole 230 may be defined as 4 mm. According to another embodiment of the present disclosure, the diameter of the side detection hole may be smaller than 4 mm or larger than 4 mm.

According to the exemplary embodiment of the present disclosure, the side detection holes 230 may include a first side hole 232 provided between the peripheral detection hole 220 and the tip connection part 120 and a second side hole 234 provided between the first side hole 232 and the tip connection part 120 and spaced apart from the first side hole 232.

For example, the first side hole 232 and the second side hole 234 may be disposed in different rows in the longitudinal direction of the tip connection part 120.

For reference, in the embodiment of the present disclosure illustrated and described above, an example is described in which the side detection holes 230 include the first side hole 232 and the second side hole 234. However, according to another embodiment of the present disclosure, the side detection holes may include three or more side holes, or the side detection hole may be provided as a single side hole.

According to an exemplary embodiment of the present disclosure, a center of the first side hole 232 may be positioned on a second reference line SL2 inclined at a preset second reference angle θ2 with respect to the centerline CL that passes through the center of the tip head part 110 and the center of the reference detection hole 210.

The second reference angle θ2 may be variously changed in accordance with required conditions and design specifications. Embodiments of the present disclosure are not restricted or limited by the size of the second reference angle θ2.

According to an exemplary embodiment of the present disclosure, the second reference angle θ2 may be defined as 90 degrees.

In addition, according to an exemplary embodiment of the present disclosure, a center of the second side hole 234 may be positioned on a third reference line SL3 inclined at a preset third reference angle θ3 with respect to the centerline CL that passes through the center of the tip head part 110 and the center of the reference detection hole 210.

The third reference angle θ3 may be variously changed in accordance with required conditions and design specifications. Embodiments of the present disclosure are not restricted or limited by the size of the third reference angle θ3.

According to an exemplary embodiment of the present disclosure, the third reference angle θ3 may be defined as 120 degrees.

The first side hole 232 and the second side hole 234 may be variously changed in number and arrangement pattern in accordance with required conditions and design specifications. Embodiments of the present disclosure are not restricted or limited by the number and arrangement pattern of the first side hole 232 and the second side hole 234.

According to an exemplary embodiment of the present disclosure, the plurality of first side holes 232 and the plurality of second side holes 234 may be provided to be radially disposed and spaced apart from one another in the circumferential direction based on the centerline CL (based on the reference detection hole 210).

In particular, four first side holes 232 may be provided to be spaced apart from one another at equal intervals in the circumferential direction based on the centerline CL, and three second side holes 234 may be provided to be spaced apart from one another at equal intervals in the circumferential direction based on the centerline CL.

According to another embodiment of the present disclosure, the number of first side holes may be defined to be smaller than four or equal to or larger than five. Likewise, the number of second side holes may be defined to be smaller than three or equal to or larger than four.

A ratio of a total sum of opening areas of the reference detection hole 210, the peripheral detection holes 220, and the side detection holes 230 to the overall area of the tip head part 110 (the overall outer surface area of the tip head part 110) may be variously changed in accordance with required conditions and design specifications.

According to an exemplary embodiment of the present disclosure, a total sum of opening areas of the reference detection hole 210, the peripheral detection holes 220, and the side detection holes 230 (e.g., a total sum of opening areas of one reference detection hole, six peripheral detection holes, four first side holes, and three second side holes) may be defined to be less than 15% of the overall area of the tip head part 110.

This is based on the fact that when the total sum of opening areas of the reference detection hole 210, the peripheral detection holes 220, and the side detection holes 230 is equal to or more than 15% of the overall area of the tip head part 110, the measurement dispersion related to the leak rate of hydrogen (gas leak value) is increased by an external influence (e.g., outside air), which degrades the detection accuracy. According to an embodiment of the present disclosure, because the total sum of opening areas of the reference detection hole 210, the peripheral detection holes 220, and the side detection holes 230 is defined to be less than 15% of the overall area of the tip head part 110, it is possible to obtain an advantageous effect of reducing the measurement dispersion related to the leak rate of hydrogen (gas leak value) caused by the external influence and improving the detection accuracy.

In particular, the total sum of opening areas of the reference detection hole 210, the peripheral detection holes 220, and the side detection holes 230 may be defined as 14% of the overall area of the tip head part 110.

Meanwhile, according to an exemplary embodiment of the present disclosure, a negative pressure may be applied to the hydrogen detection line 30, and a suction pressure may be applied to the reference detection hole 210, the peripheral detection holes 220, and the side detection holes 230 on the basis of the negative pressure.

A suction rate (suction rate implemented by the suction pressure) through the reference detection hole 210, the peripheral detection holes 220, and the side detection holes 230 may be variously changed in accordance with required conditions and design specifications. Embodiments of the present disclosure are not restricted or limited by the suction rate through the reference detection hole 210, the peripheral detection holes 220, and the side detection holes 230.

According to an exemplary embodiment of the present disclosure, the suction rate through the reference detection hole 210, the peripheral detection holes 220, and the side detection holes 230 may be defined as 3,000 sccm.

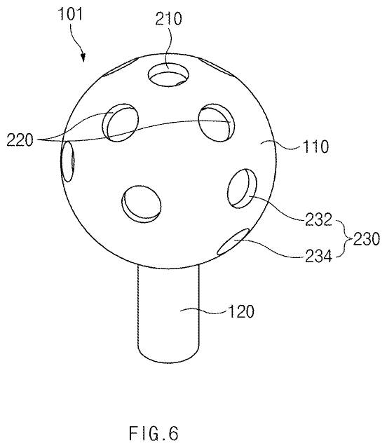

Hereinafter, comparative examples (Comparative Examples 1 to 4) of the gas leak detection device according to an embodiment of the present disclosure will be described with reference to FIGS. 6 to 9.

With reference to FIG. 6, Comparative Example 1 (101) of the gas leak detection device according to an embodiment of the present disclosure includes the tip head part 110 (diameter of 20 mm, thickness of 2 mm) and the tip connection part 120. The tip head part 110 has a total of fifteen detection holes including the reference detection hole 210 (diameter of 4 mm), the peripheral detection holes 220 (diameter of 4 mm), and the side detection holes 230 (diameter of 4 mm), and the total sum of opening areas of the reference detection hole 210, the peripheral detection holes 220, and the side detection holes 230 is defined as 15% of the overall area of the tip head part 110. In addition, in Comparative Example 1 (101), the tip detection zone is defined as 22% of the overall area of the tip head part, and the total sum of opening areas of the reference detection hole 210 and the peripheral detection holes 220 is defined as 22.7% of the area of the tip detection zone.

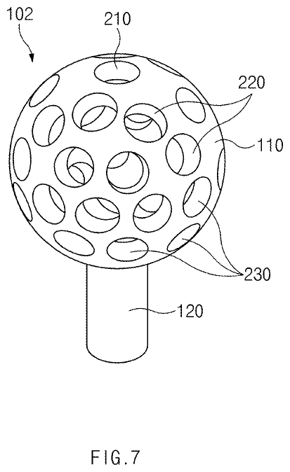

With reference to FIG. 7, Comparative Example 2 (102) of the gas leak detection device according to an embodiment of the present disclosure includes the tip head part 110 (diameter of 20 mm, thickness of 2 mm) and the tip connection part 120. The tip head part 110 has a total of forty-five detection holes including the reference detection hole 210 (diameter of 4 mm), the peripheral detection holes 220 (diameter of 4 mm), and the side detection holes 230 (diameter of 4 mm), and the total sum of opening areas of the reference detection hole 210, the peripheral detection holes 220, and the side detection holes 230 is defined as 45% of the overall area of the tip head part 110.

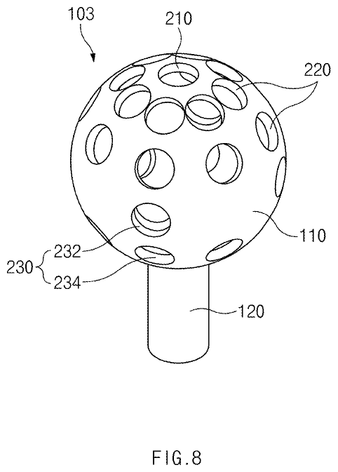

With reference to FIG. 8, Comparative Example 3 (103) of the gas leak detection device according to an embodiment of the present disclosure includes the tip head part 110 (diameter of 20 mm, thickness of 2 mm) and the tip connection part 120. The tip head part 110 has a total of seventeen detection holes including the reference detection hole 210 (diameter of 4 mm), the peripheral detection holes 220 (diameter of 4 mm), and the side detection holes 230 (diameter of 4 mm), and the total sum of opening areas of the reference detection hole 210, the peripheral detection holes 220, and the side detection holes 230 is defined as 17% of the overall area of the tip head part 110. In addition, in Comparative Example 3 (103), the tip detection zone is defined as 33.7% of the overall area of the tip head part, and the total sum of opening areas of the reference detection hole 210 and the peripheral detection holes 220 is defined as 50.4% of the area of the tip detection zone.

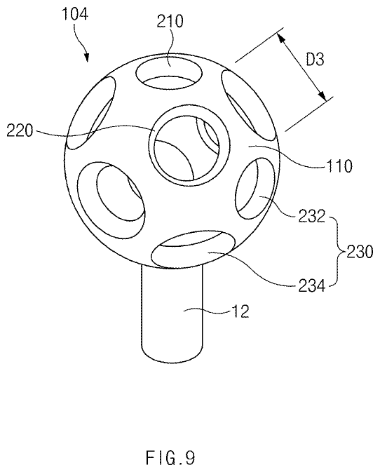

With reference to FIG. 9, Comparative Example 4 (104) of the gas leak detection device according to an embodiment of the present disclosure includes the tip head part 110 (diameter of 20 mm, thickness of 2 mm) and the tip connection part 120. The tip head part 110 has a total of fourteen detection holes including the reference detection hole 210 (diameter of 8 mm) (D3), the peripheral detection holes 220 (diameter of 8 mm) (D3), and the side detection holes 230 (diameter of 8 mm) (D3), and the total sum of opening areas of the reference detection hole 210, the peripheral detection holes 220, and the side detection holes 230 is defined as 52.5% of the overall area of the tip head part 110.

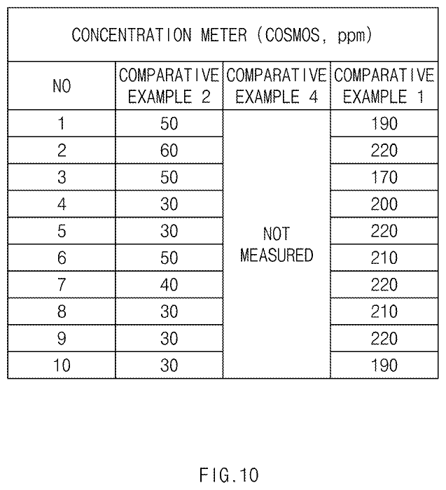

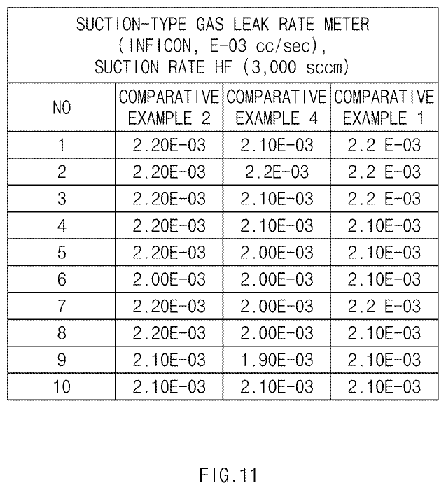

FIG. 10 is a view for explaining the gas leak values of the comparative examples in accordance with the number of detection holes and the sizes of the detection holes, and FIG. 11 is a view for explaining the gas leak rates of the comparative examples in accordance with the number of detection holes and the sizes of the detection holes.

For reference, FIG. 10 illustrates a result of measuring the concentrations (gas leak values) of hydrogen leaking (basic gas leak value: 2.4 to 2.5 E-03 cc/sec) from the detection target (hydrogen leak site) for respective comparative examples by using a concentration meter of COSMOS. FIG. 11 illustrates a result of measuring the gas leak rates of hydrogen leaking (basic gas leak value: 2.4 to 2.5 E-03 cc/sec) from the detection target (hydrogen leak site) for respective comparative examples by using a suction-type gas leak rate meter of INFICON.

With reference to FIG. 10, it can be seen that as the sizes (diameters) and the number of the detection holes (the reference detection hole, the peripheral detection holes, and the side detection holes) provided in the tip head part 110 are increased, the detection rate decreases, and the dispersion increases, which makes it difficult to establish criteria.

In particular, it can be seen that in Comparative Example 2 (forty-five detection holes with a detection hole diameter of 4 mm) 102, the gas leak value is small, and the detection rate decreases in comparison with Comparative Example 1 (fifteen detection holes with a detection hole diameter of 4 mm) 101. In addition, it can be seen that in Comparative Example 4 (fourteen detection holes with a detection hole diameter of 8 mm) 104, the detection rate significantly decreases, and the gas leak value cannot be measured (the gas leak value is present as a lower limit value of the detection range) in comparison with Comparative Examples 1 and 2. Therefore, it can be seen that a small size and number of detection holes is relatively advantageous for measurement dispersion.

In contrast, with reference to FIG. 11, it can be seen that because the use of the suction-type gas leak rate meter compensates for a suction force related to the measurement dispersion, the measurement dispersion is comparatively small for the comparative examples (Comparative Examples 1, 2, and 4).

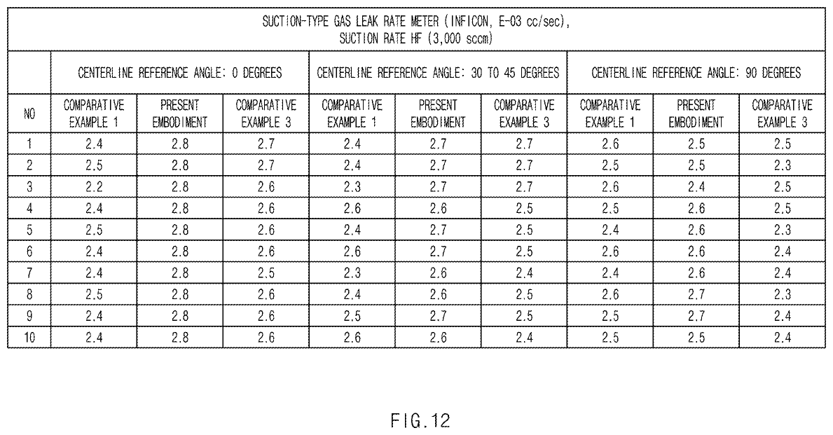

FIG. 12 is a view for explaining the gas leak rates of embodiments of the present disclosure in accordance with detection angles with respect to leak points.

For reference, FIG. 12 illustrates a result of measuring the gas leak rates of hydrogen leaking (basic gas leak value: 2.7 to 2.8 E-03 cc/sec) from the detection target (hydrogen leak site) in accordance with the detection angles with respect to the leak points for embodiments of the present disclosure and the comparative examples by using the suction-type gas leak rate meter of INFICON.

With reference to FIG. 12, it can be seen that in embodiments of the present disclosure, the dispersion in accordance with the detection angle with respect to the leak point (an angle of the leak point with respect to the tip head part based on the centerline) is comparatively small. In particular, it can be seen that in embodiments of the present disclosure, the accuracy of the gas leak value (2.8 E-03 cc/sec) is highest at a point at which the detection angle with respect to the leak point is 0 degrees (at a portion of the reference detection hole 210).

In contrast, it can be seen that in Comparative Example 1 (101), the accuracy of the gas leak value (2.4 to 2.5 E-03 cc/sec) at the point at which the detection angle with respect to the leak point is o degrees is lower than that of embodiments of the present disclosure. Further, it can be seen that in Comparative Example 3 (103), the ratio of the tip detection zone to the overall area of the tip head part is higher than that of embodiments of the present disclosure (Comparative Example 3: 33.7% vs. Present Embodiment: 12.3%), and the ratio of the total sum of the opening areas of the reference detection hole 210 and the peripheral detection holes 220 to the area of the tip detection zone is lower than that of embodiments of the present disclosure (Comparative Example 3: 50.4% vs. Present Embodiment: 57.1%), such that the dispersion (2.3 to 2.5 E-03 cc/sec) of the gas leak value is large because of the great influence of outside air.

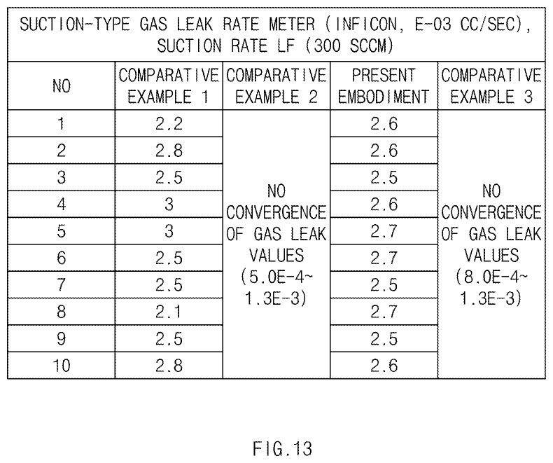

FIG. 13 is a view for explaining the gas leak rates of embodiments of the present disclosure in accordance with the number of detection holes under a low-suction rate condition.

For reference, FIG. 13 illustrates a result of measuring the gas leak rate of hydrogen leaking from the detection target (hydrogen leak site) under a low-suction rate condition of 300 sccm by using the suction-type gas leak rate meter of INFICON.

With reference to FIG. 13, it can be seen that under the low-suction rate condition, the increase in the number of detection holes (the reference detection hole, the peripheral detection holes, and the side detection holes) makes it difficult to converge the gas leak values.

In particular, it can be seen that in Comparative Example 1 (fifteen detection holes with a detection hole diameter of 4 mm) 101, the dispersion of the gas leak values is larger than that of an embodiment of the present disclosure (fourteen detection holes with a detection hole diameter of 4 mm). Further, it can be seen that in Comparative Example 2 (forty-five detection holes with a detection hole diameter of 4 mm) 104 and Comparative Example 3 (seventeen detection holes with a detection hole diameter of 4 mm) 103, the gas leak values cannot be converged.

Therefore, it can be seen that the ratio of the tip detection zone to the overall area of the tip head part and the ratio of the total sum of the opening areas of the reference detection hole and the peripheral detection holes to the area of the tip detection zone affect the gas leak rate.

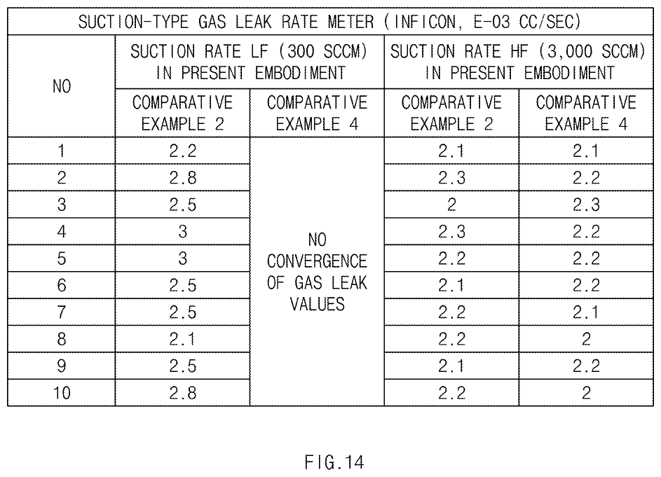

FIG. 14 is a view for explaining the gas leak rates of the comparative examples in accordance with the sizes of the detection holes under a low-suction rate condition and a high-suction rate condition.

For reference, FIG. 14 illustrates a result of measuring the gas leak rates of hydrogen leaking from the detection target (hydrogen leak site) under a high-suction rate condition of 3,000 sccm and a low-suction rate condition of 300 sccm by using the suction-type gas leak rate meter of INFICON.

With reference to FIG. 14, it can be seen that under the low-suction rate condition of 300 sccm, it is difficult to converge the gas leak values in Comparative Example 2 (forty-five detection holes with a detection hole diameter of 4 mm) 102 and Comparative Example 4 (fourteen detection holes with a detection hole diameter of 8 mm) 104 in which the number and size (diameter) of the detection holes (the reference detection hole, the peripheral detection holes, and the side detection holes) are larger than those of embodiments of the present disclosure.

In contrast, it can be seen that under the high-suction rate condition of 3,000 sccm, as in Comparative Example 2 (102) and Comparative Example 4 (104), the gas leak values may be converged even when the number and size (diameter) of the detection holes (the reference detection hole, the peripheral detection holes, and the side detection holes) are increased, but the amount of time required to converge the gas leak values increases, and the dispersion of the gas leak values (2.0 to 2.3 E-03 cc/sec) is comparatively large.

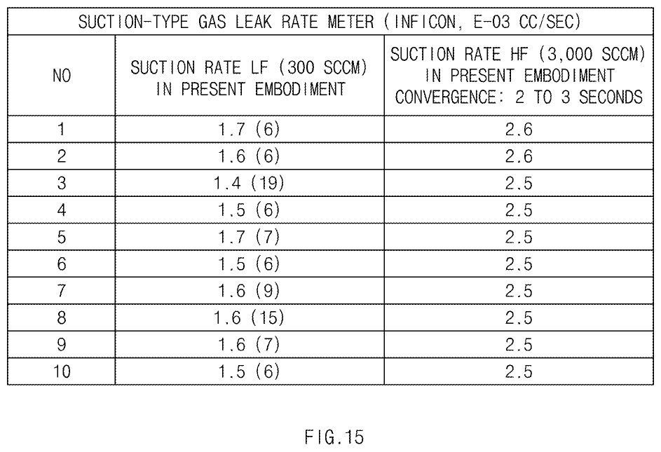

Meanwhile, FIG. 15 is a view for explaining the gas leak rates of embodiments of the present disclosure in accordance with suction rates.

For reference, FIG. 15 illustrates a result of measuring the gas leak rates of hydrogen leaking from the detection target (hydrogen leak site) under a high-suction rate condition of 3,000 sccm and a low-suction rate condition of 300 sccm by using the suction-type gas leak rate meter of INFICON.

With reference to FIG. 15, it can be seen that in embodiments of the present disclosure, under the low-suction rate condition of 300 sccm, the detection rate decreases, and a relatively large amount of time is required to converge the gas leak values in comparison with the high-suction rate condition of 3,000 sccm.

In particular, it can be seen that under the high-suction rate condition of 3,000 sccm, the detection rate (2.5 to 2.6 E-03 cc/sec) is high, and the amount of time required to converge the gas leak values is small (2 to 3 seconds), whereas under the low-suction rate condition of 300 sccm, the detection rate (1.4 to 1.7 E-03 cc/sec) is low, and the amount of time required to converge the gas leak values increases (6 to 19 seconds).

According to embodiments of the present disclosure described above, it is possible to obtain an advantageous effect of improving the accuracy of the hydrogen leak inspection and improving the safety and reliability.

In particular, according to embodiments of the present disclosure, it is possible to obtain an advantageous effect of minimizing a deviation in the accuracy of the hydrogen leak inspection, which varies depending on the operator's experience and competence, and easily and accurately detecting whether hydrogen leaks.

Among other things, according to embodiments of the present disclosure, it is possible to obtain an advantageous effect of accurately performing the hydrogen leak inspection without being greatly affected by the posture (angle and distance) of the detection tip with respect to the inspection site.

In addition, according to embodiments of the present disclosure, it is possible to obtain an advantageous effect of simplifying the hydrogen leak inspection process and shortening the time required for the inspection process.

In addition, according to embodiments of the present disclosure, it is possible to obtain an advantageous effect of minimizing an error in the hydrogen leak inspection and quantifying the result of the hydrogen leak inspection.

While the embodiments have been described above, the embodiments are just illustrative and are not intended to limit the present disclosure. It can be appreciated by those skilled in the art that various modifications and applications, which are not described above, may be made to the present embodiments without departing from the intrinsic features of the present embodiments. For example, the respective constituent elements specifically described in the embodiments may be modified and then carried out. Further, it should be interpreted that the differences related to the modifications and applications are included in the scope of the present disclosure defined by the appended claims.

What is claimed is:

1. A gas leak detection device comprising:
a tip head part having a spherical shape;
a tip connection part configured to communicate with the tip head part and connectable to a hydrogen detection line;
a reference detection hole disposed at an end of the tip head part based on a longitudinal direction of the tip connection part; and
a peripheral detection hole disposed in the tip head part, spaced apart from the reference detection hole, and directed in a direction intersecting the reference detection hole.

2. The gas leak detection device of claim 1, wherein a center of the peripheral detection hole is positioned on a first reference line inclined at a preset first reference angle with respect to a centerline that passes through a center of the tip head part and a center of the reference detection hole.

3. The gas leak detection device of claim 2, wherein the first reference angle is 45 degrees or less.

4. The gas leak detection device of claim 2, wherein the first reference angle is 30 degrees.

5. The gas leak detection device of claim 2, wherein the peripheral detection hole comprises a plurality of peripheral detection holes spaced apart from one another in a circumferential direction based on the centerline.

6. The gas leak detection device of claim 2, wherein:
the tip head part has a diameter of 20 mm and a thickness of 2 mm;
a diameter of each of the reference detection hole and the peripheral detection hole is 4 mm; and
the peripheral detection hole comprises seven peripheral detection holes spaced apart from one another at equal intervals in a circumferential direction based on the centerline.

7. A gas leak detection device comprising:
a tip head part having a spherical shape;
a tip connection part configured to communicate with the tip head part and connectable to a hydrogen detection line;
a reference detection hole disposed at an end of the tip head part based on a longitudinal direction of the tip connection part;
a peripheral detection hole disposed in the tip head part, spaced apart from the reference detection hole, and directed in a direction intersecting the reference detection hole; and
side detection holes disposed in the tip head part and positioned between the peripheral detection hole and the tip connection part.

8. The gas leak detection device of claim 7, wherein the side detection holes comprise:
a first side hole disposed between the peripheral detection hole and the tip connection part; and
a second side hole disposed between the first side hole and the tip connection part and spaced apart from the first side hole.

9. The gas leak detection device of claim 8, wherein:
a center of the first side hole is positioned on a second reference line inclined at a preset second reference angle with respect to a centerline that passes through a center of the tip head part and a center of the reference detection hole; and
a center of the second side hole is positioned on a third reference line inclined at a preset third reference angle with respect to the centerline that passes through the center of the tip head part and the center of the reference detection hole.

10. The gas leak detection device of claim 9, wherein the second reference angle is 90 degrees and the third reference angle is 120 degrees.

11. The gas leak detection device of claim 9, wherein:
the first side hole comprises a plurality of first side holes spaced apart from one another in a circumferential direction based on the centerline; and
the second side hole comprises a plurality of second side holes spaced apart from one another in the circumferential direction based on the centerline.

12. The gas leak detection device of claim 11, wherein:
the tip head part has a diameter of 20 mm and a thickness of 2 mm;
a diameter of each of the plurality of first side holes and the plurality of second side holes is 4 mm;
the plurality of first side holes comprises four first side holes spaced apart from one another at equal intervals in the circumferential direction based on the centerline; and
the plurality of second side holes comprises three second side holes spaced apart from one another at equal intervals in the circumferential direction based on the centerline.

13. The gas leak detection device of claim 7, wherein a total sum of opening areas of the reference detection hole, the peripheral detection hole, and the side detection holes is less than 15% of an overall area of the tip head part.

14. The gas leak detection device of claim 7, wherein in a case in which a negative pressure is applied to the hydrogen detection line, a suction pressure is applied to the reference detection hole, the peripheral detection hole, and the side detection holes based on the negative pressure.

15. The gas leak detection device of claim 14, wherein a suction rate through the reference detection hole, the peripheral detection hole, and the side detection holes is 3,000 sccm.

16. A gas leak detection device comprising:
a tip head part having a spherical shape;
a tip connection part configured to communicate with the tip head part and connectable to a hydrogen detection line;
a reference detection hole disposed at an end of the tip head part based on a longitudinal direction of the tip connection part; and
a peripheral detection hole disposed in the tip head part, spaced apart from the reference detection hole, and directed in a direction intersecting the reference detection hole, wherein the reference detection hole and the peripheral detection hole are positioned in a tip detection zone on a tip portion of the tip head part based on the longitudinal direction of the tip connection part.

17. The gas leak detection device of claim 16, wherein the tip detection zone is 15% or less of an overall area of the tip head part.

18. The gas leak detection device of claim 16, wherein a total sum of opening areas of the reference detection hole and the peripheral detection hole is 55% or more of an area of the tip detection zone.

19. The gas leak detection device of claim 16, wherein a total sum of opening areas of the reference detection hole and the peripheral detection hole is 57.1% of an area of the tip detection zone.

20. The gas leak detection device of claim 16, wherein a center of the peripheral detection hole is positioned on a first reference line inclined at a preset first reference angle with respect to a centerline that passes through a center of the tip head part and a center of the reference detection hole.
